(19) United States
(12) Patent Application Publication
(10) Pub. No.: US 2026/0283648 A1
(43) Pub. Date: Sep. 24, 2026

(54) DEVICE FOR FIXING A CRANIAL FLAP TO THE CRANIAL VAULT TO BE POSITIONED IN THE CRANIOTOMY HOLE

(71) Applicant: NTPLAST S.R.L., Rome (IT)

(72) Inventor: Igino Romolo GAZZANI, Rome (IT)

(21) Appl. No.: 19/470,048

(22) PCT Filed: Mar. 18, 2024
(86) PCT No.: PCT/IT2024/050057

§ 371 Date: Sep. 28, 2025

(30) Foreign Application Priority Data
Mar. 31, 2023 (IT) 102023000006270

(51) Int. Cl. A61B 17/68; A61B 17/00; A61B 17/80

(52) U.S. Cl. CPC A61B 17/688; A61B 17/683; A61B 17/8061; A61B 17/808; A61B 2017/00473; A61B 2017/00862

(57) ABSTRACT

A device (10) is described for fixing a cranial flap to the cranial vault (17) capable of being positioned in a craniotomy hole, comprising: a cortical support upright (12); first closing means (11, 13, 13′) operatively connected to the upright (12); second closing means (14) to be fixed to the upright (12) to complete the closing; a handle (15) connected to the cortical upright (12); and a base (5) for better cooperation with the elastic arms (11); the first closing means (11, 13, 13′) are made up of at least two elastic arms (11) operatively connected to the cortical upright (12) and which each end in at least one curved transversal arm (13′).

The present invention refers to a device for fixing a cranial flap to the cranial vault to be positioned in the craniotomy hole. A dedicated applicator for this device and a process for using this device are also described.

The present invention constitutes an improvement of the devices described in documents WO-A1-2009/044421 and EP-A1-2 988 687 in the name of the same Applicant.

Document US-A1-2008281339 discloses a device according to the preamble of claim 1.

As is known, craniotomy, i.e. the incision and cutting of a bony flap of the cranial vault, is the mandatory neurosurgical procedure for the treatment of any intracranial lesion.

A bone flap is carved by making one or more drill holes, according to two preferred procedures. The first involves using a cutting blade or a perforator applied to a pneumatic drill, which, freehand, detaches the underlying dura mater from the key hole and simultaneously cuts into the bone. The second procedure, however, involves dissecting the dura mater from the bone between one drill hole and another with a curved periosteal detacher and subsequently, using a guide, passing a saw wire, which, pulled wide by the two ends upward with hands, carves the bone one segment at a time.

At the end of the neurosurgical operation, after having sutured the dura mater and suspended the edges to the bone, the flap is placed back into the opening and fixed with detached metal or wire stitches passed through small drill holes paired on the free edge of the cranial bone.

It appears clear, however, that a solution of this type does not generally allow an aesthetically acceptable closing, since it is not always able to prevent the bone flap from surfacing, sinking, tilting or rotating.

Object of the present invention is, therefore, solving the aforementioned prior art problems, creating a device for fixing a cranial flap to the cranial vault which allows the correct ossification of the bone flap at the edge of the cranial vault and at the same time closes the holes made for craniotomy.

Another object of the invention is creating a device that allows for less tension on the brain itself, even in the event that a cerebral edema occurs during the postoperative course.

Compared to the devices described in WO-A1-2009/044421 and EP-A1-2 988 687 mentioned above, which necessarily require the presence of drill holes to be positioned, the present invention can be inserted indiscriminately into the craniotomy hole or into the cut craniotomy, thus allowing the flap to be fixed in multiple points even when only one hole has been made, or the latter has an irregular shape. Furthermore, the special shape of the transverse arms allows for a better grip on the cranial bone and therefore better fixation of the flap.

A further object of the present invention is creating a lower base to be assembled on the device indicated above to improve its functionality in the following ways:
increase resistance to compressive loads that could act on the perculum after closing the craniotomy (e.g. accidental impacts that the patient could suffer), due to a greater support surface;
allow the elastic arms of the device to slide easily during the positioning step, regardless of the presence of irregularities in the internal surface of the skull; and
distribute loads more evenly on the internal surface of the skull.

As an optional feature, the base can be equipped with a centring device whereby, when the device is positioned inside a drill hole, a better centring of the operculum in the craniotomy is achieved.

These and other objects are achieved by a device for fixing a cranial flap to the cranial vault to be positioned in the craniotomy hole as described in claim 1.

Further characteristics of the invention are defined in the dependent claims.

It is understood that all claims form an integral part of this description.

Further objects and advantages of the present invention will be clear from the following description and from the attached drawings, provided purely by way of example and not by way of limitation, in which:

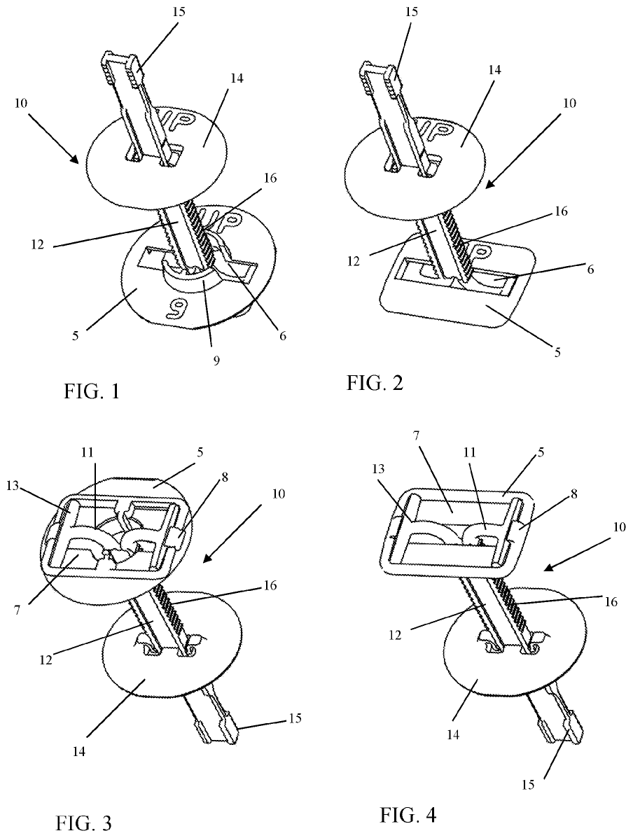

FIG. 1 shows a perspective view from above of a first example of the device for fixing a cranial flap to the cranial vault to be positioned in the craniotomy hole or in the craniotomy cut;

FIG. 2 shows a perspective view from above of a second example of the device for fixing a cranial flap to the cranial vault to be positioned in the craniotomy hole or in the craniotomy cut;

FIG. 3 is a perspective view from below of the device of FIG. 1;

FIG. 4 is a perspective view from below of the device of FIG. 2;

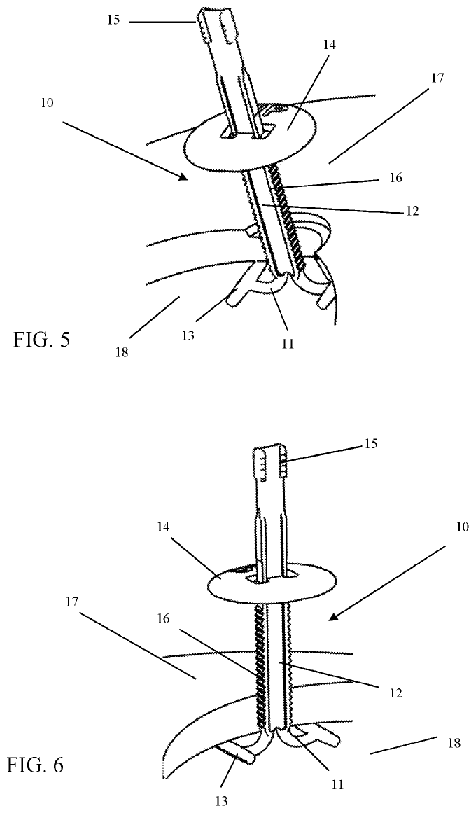

FIG. 5 shows a view of the device of FIG. 1 or 2 in a first step of its use in the craniotomy hole;

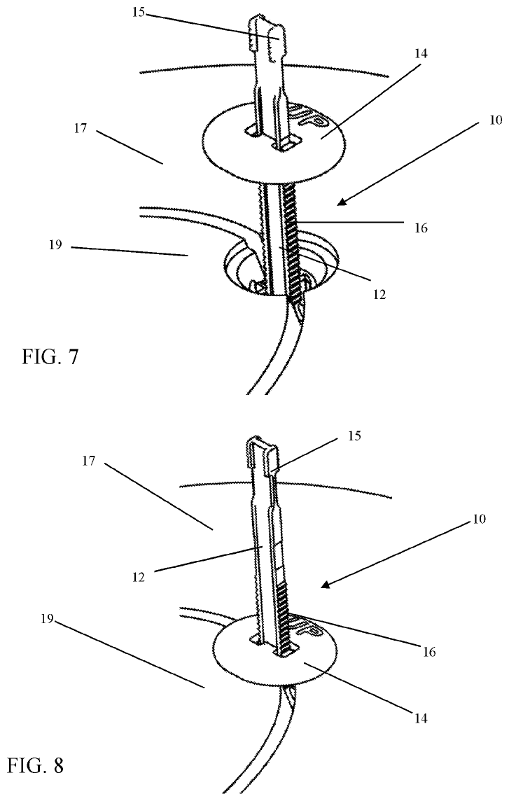

FIG. 6 shows a view of the device of FIG. 1 or 2 in a first step of its use in the craniotomy cutting;

FIG. 7 shows a view of the device of FIG. 1 or 2 in a second step of its use in the craniotomy cutting;

FIG. 8 shows a view of the device of FIG. 1 or 2 in a second step of its use in the craniotomy cutting;

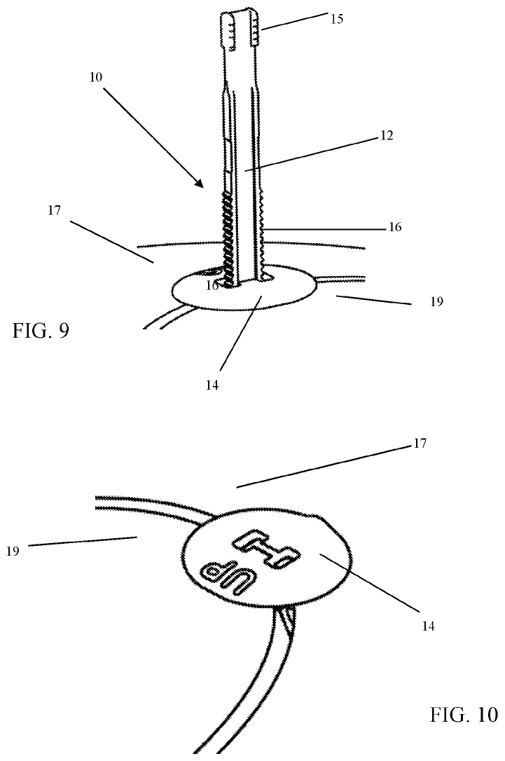

FIGS. 9 and 10 show views of the device in FIG. 1 or 2 in the final stages of its use in the craniotomy cutting;

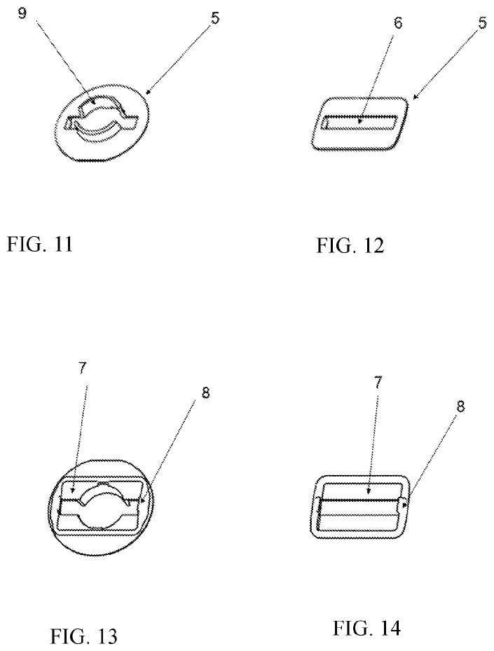

FIGS. 11 and 13 show a view from above and one from below (the latter which better highlights the fixing system of the elastic arms of the device in FIG. 1), respectively, of a first embodiment of the base to be used with the device of FIG. 1;

FIGS. 12 and 14 show a view from above and one from below (the latter which better highlights the fixing system of the elastic arms of the device in FIG. 1), respectively, of a second embodiment of the base to be used with the device of FIG. 2;

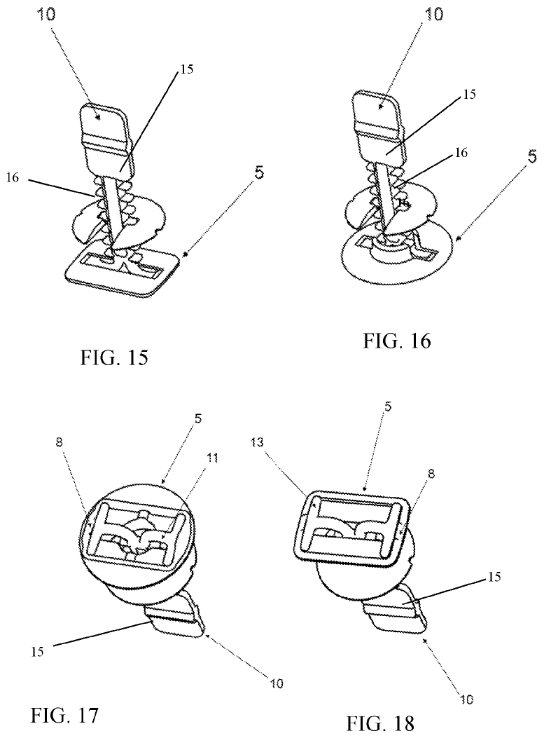

FIGS. 15 and 17 show a top and bottom view, respectively, of a third example of the fixing device;

FIGS. 16 and 18 represent a top and bottom view, respectively, of a fourth example of the fixing device;

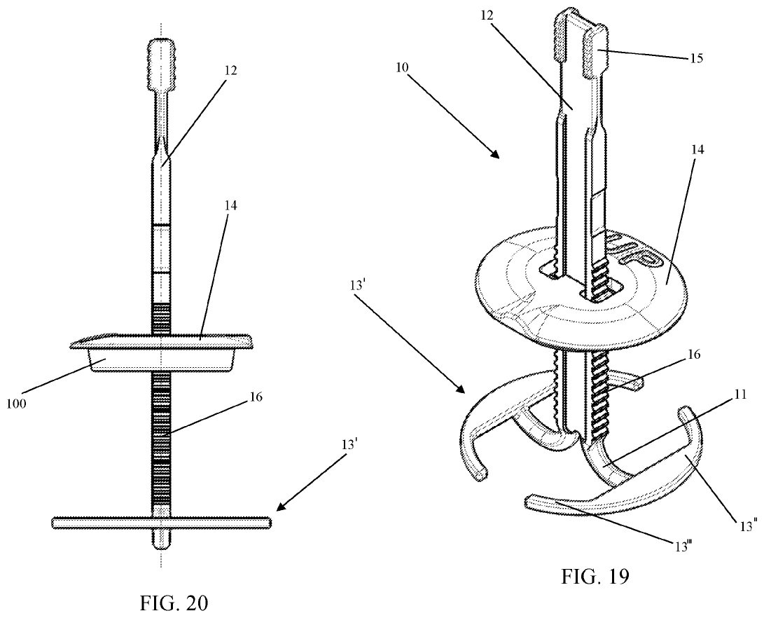

FIG. 19 represents a perspective view of an embodiment of the device of the present invention; and

FIG. 20 represents a side view of the device of FIG. 19.

With particular reference to the figures, the device for fixing a cranial flap to the cranial vault to be positioned in the craniotomy hole or in the craniotomy cut, according to the present invention, is globally indicated with numeric reference 10.

The device 10 has elongated elastic arms 11, linked to the cortical upright 12, and ending in elongated arms 13, 13′ placed transversely with respect to the axis of the elongated elastic arms 11, and an upper plate 14, which must be inserted in the upper part of the cortical upright 12.

The cortical upright 12 will be equipped with a handle 15 and a plurality of fixing elements 16 (which can be, for example, teeth as illustrated in FIGS. 1 to 9, or transversal protrusions as illustrated in FIGS. 15 to 18) placed at as many levels along the length of the upright 12, fixing elements 16 which will be used for the insertion of the upper plate 14.

The cortical upright 12, connecting the elastic arms 11 and the upper plate 14, due to the elasticity of the arms 11, allows a fine adaptation to the thickness of the bone.

The conformation of the inventive device 10 therefore provides that the first closing means 11, 13, 13′ are connected to the distal end of the cortical upright 12 and the second closing means 14 are suitable for being fixed to the cortical upright 12 in a nearby position of the distal end of the cortical upright 12, such that, when the device 10 is in its operative fixing position, the first closing means 11, 13, 13′ are placed against the internal distal surface of the skull and the second closing means 14 are placed against the near outer surface of the skull.

Summarizing in detail, therefore, as illustrated in the Figures and in particular in FIG. 19, the device 10 of the invention serves for fixing a cranial flap to the cranial vault 17 capable of being positioned in a craniotomy hole, and includes:
at least one cortical support upright 12;
first closing means 11, 13, 13′ operatively connected to the cortical upright 12;
second closing means 14 suitable for being fixed to the cortical upright 12 to complete its closing;
at least one handle 15 connected in a removable manner to the cortical upright 12 and suitable for driving the device 10 to bring it from its rest position to its operational fixing position;
wherein the first closing means 11, 13, 13′ are connected to the distal end of the cortical upright 12 and the second closing means 14 are adapted to be fixed to the cortical upright 12 in a position close to the distal end of the cortical upright 12, such that, when the device 10 is in its operative fixing position, the first closing means 11, 13, 13′ are placed against the internal distal surface of the skull and the second closing means 14 are placed against the near external surface of the skull, the first closing means 11, 13, 13′ being composed of at least two elongated elastic arms 11 operatively connected to the cortical upright 12, each of the elongated elastic arms 11 ending in an elongated arm 13, 13′ placed transversely with respect to the elongated elastic arms 11,
wherein the device 10 is also equipped with at least one lower base 5 to be assembled to the device 10 for fixing a cranial flap and for simultaneously closing the holes made to perform the craniotomy, the lower base 5 having a cut 6 through which the device 10 passes when the device 10 moves into its operative fixing position, and a seat 7 where the transverse arms 13, 13′ linked to the elastic arms 11 are housed and slide.

The first feature of the present invention is that the transverse elongated arms 13′ are substantially semi circular in shape with greater transverse thickness in their central part 13″ compared to their two ends 13″′, each of the transverse elongated arms 13′ having their ends 13″′ oriented towards the respective ends 13″′ of the other of the elongated transverse arms 13′ so as to leave a space between the ends 13″′, as can be clearly seen in FIG. 19.

Another feature of the present invention is that the second closing means 14 is equipped with a cylindrical collar 100 designed to centre the second closing means 14 in the craniotomy hole of the skull during installation.

Going into more detail into the methods of application of the cranial fixation device 10 of the invention, it should be noted that the instrumentalist prepares the quantity of devices 10 suitable for fixing the craniotomy.

These operations are carried out by choosing the most suitable number of devices 10, in relation to the size and shape of the craniotomy.

To use the device 10 in practice, an applicator (not illustrated) suitable for operating with the device 10 described above is provided, preferably but not exclusively; this applicator includes a seat designed to support the upper plate 14 and to allow the operational coupling between this plate 14 and the cortical upright 12 at the end of the closing of the craniotomy hole or the craniotomy cut.

To position the device in the craniotomy hole, the following operations are required.

The surgeon inserts the device 10 into the craniotomy hole (FIG. 5) with the cortical upright 12 placed in the centre of the hole and perpendicular to the cranial surface, and so that part of the transverse arms 13, 13′ is positioned in the space between the cranial vault 17 and the dura mater 18.

The bony operculum 19 is repositioned as indicated in FIG. 7. In this way the cortical upright 12 will protrude from the craniotomy hole.

Subsequently, the surgeon checks the good position of the operculum 19 and relaxes, using the handle 15, the cortical upright 12 and the elastic arms 11 to be able to insert the upper plate 14 in correspondence with one of the fixing elements 16, as shown in the FIG. 9. When inserting the upper plate 14, the surgeon uses the applicator described above. In this way, the craniotomy hole will be closed. Finally, the surgeon removes the handle 15 with nippers (FIG. 10).

To position the device in the craniotomy cut, the following operations are required.

The surgeon inserts the device 10 into the craniotomy cut: (FIG. 6) so that one end of the transverse arms 13, 13′ is positioned in the space between the cranial vault 17 and the dura mater 18, with the elastic arms 11 aligned to the direction of the cut craniotomy and the cortical upright 12 perpendicular to the cranial surface.

The bony operculum 19 is placed on the other ends of the transverse arms 13 of the device 10, as indicated in FIG. 8. In this way, the upright 12 will protrude between the cranial vault 17 and the operculum 19.

Subsequently, the surgeon checks the good position of the operculum 19 and relaxes, using the handle 15, the cortical upright 12 and the elastic arms 11 to be able to insert the upper plate 14 in correspondence with one of the fixing elements 16, as shown in the FIG. 9. When inserting the upper plate 14, the surgeon uses the applicator described above. Finally, the surgeon removes the handle 15 with nippers (FIG. 10).

The fixing device 10 also includes, as illustrated in the various Figures and in detail in FIGS. 11 to 14 in two preferred, but not limiting, examples, a lower base 5, preferably made of biocompatible elastic plastic material, to be assembled to the device 10 for the fixing of a cranial flap and for the simultaneous closing of the holes made to perform the craniotomy.

With particular reference to FIGS. 11 to 14, the lower base is indicated globally with numeric reference 5; the device for fixing a cranial flap to the cranial vault and the simultaneous closing of the craniotomy holes, as Seen previously, is indicated globally with numeric reference 10.

The lower base 5 has the cut 6 through which the device 10 can pass, and a seat 7 where the transverse arms 13, 13′ linked to the elastic arms 11 are housed and slide. In this way, through the sliding of the transversal arms 13, 13′ during the positioning step of the device 10, regardless of the presence of irregularities in the internal surface of the skull, a better application functionality of the device 10 itself is obtained.

The transverse arms 13, 13′ are blocked, preventing them from opening in an excessive and unusable way, by the very shape of the seat 7 of the base 5, or by special coupling means 8 provided at two ends of the seat 7 (as seen in FIGS. 3, 4, 17 and 18).

A second version of the lower base 5, illustrated in FIGS. 1, 11 and 16, has a centring system 9, designed for the use of the device 10 in the holes made by the drill.

The cranial fixing device 10 of the invention is indicated in all cases of craniotomy, as its shape and its adaptability to the bone thickness allow the bone flap to be kept adherent to the edge of the cranial vault, both on the internal margin and on the external margin, in order to allow the correct ossification.

At the same time, the elasticity of the biocompatible plastic material used guarantees less tension on the brain during the upright-operative period, when conditions of cerebral edema and therefore swelling of the brain itself may occur.

From the description given, the features of the device 10 for fixing a cranial flap to the cranial vault to be positioned in the craniotomy hole or in the craniotomy cut, which is the object of the present invention, are clear, just as the advantages are clear.

Furthermore, in the practical implementation of the invention, the materials, shapes and dimensions of the illustrated details may be any according to needs and they may be replaced with others that are technically equivalent.

Finally, it is clear that numerous variations can be made to the device 10 for fixing a cranial flap to the cranial vault to be positioned in the craniotomy hole or in the craniotomy cut, without thereby departing from the scope of the present invention, as defined in the attached claims.

1. A device for fixing a cranial flap to the cranial vault capable of being positioned in a craniotomy hole, comprising:
at least one cortical support upright;
first closing means operatively connected to the cortical upright;
second closing means suitable to be fixed to the cortical upright to complete its closing;
at least one handle connected in a removable way to the cortical upright and suitable for driving the device to bring it from its rest position to its operational fixing position;
wherein the first closing means are connected to the distal end of the cortical upright and the second closing means are adapted to be fixed to the cortical upright in a position close to the distal end of the cortical upright, such that, when the device is in its operative fixing position, the first closing means are placed against the distal surface internal part of the skull and the second closing means are placed against the near external surface of the skull, the first closing means being constituted by at least two elongated elastic arms operatively connected to the cortical upright, each of the elongated elastic arms ending in an elongated arm placed transversely with respect to the elongated elastic arms,
wherein the device is also equipped with at least one lower base to be assembled to the device for fixing a cranial flap and for simultaneously closing the holes made to perform the craniotomy, the lower base having a cut (6) through which the device passes when the device moves into its operative fixing position, and a seat where the transverse arms tied to it are housed and slide to the elastic arms,
characterized in that the transverse elongated arms are substantially semi-circular in shape with greater transverse thickness in their central part compared to their two ends, each of the transverse elongated arms (13′) having its ends oriented towards the respective ends of the other of the transverse elongated arms-(13′) so as to leave a space between the ends; and
in that the second closing means are equipped with a cylindrical collar designed to centre the second closing means in the craniotomy hole.

2. The device according to claim 1, characterized in that the transverse arms are blocked in opening by the shape of the seat of the base.

3. The device according to claim 1, characterized in that the transverse arms are blocked in opening by coupling means provided at two ends of the seat of the base.

4. The device according to claim 1, characterized in that the base has a centring system intended for use when the device is used in holes made by a drill.

5. The device according to claim 1, characterized in that it is made of elastic biocompatible plastic material.

6. The device according to claim 1, characterized in that the second closing means are composed of an upper plate suitable for being inserted in the upper part of the cortical upright.

7. The device according to claim 6, characterized in that the cortical upright is also equipped with a plurality of fixing elements placed at as many levels along the length of the upright, the fixing elements being capable of engaging the upper plate after its operational coupling with the upright.
